(19) United States
(12) Patent Application Publication
(10) Pub. No.: US 2026/0248993 A1
(43) Pub. Date: Aug. 27, 2026

(54) PORTABLE FLUID COLLECTION SYSTEMS WITH STORAGE AND RELATED METHODS

(71) Applicant: PureWick Corporation, Covington, GA (US)

(72) Inventors: Serena Agrawal, Atlanta, GA (US); Adam Martin, Holly Springs, NC (US); Stephanie Metzger, Atlanta, GA (US); Gina Whitlock, Barrington Hills, IL (US)

(21) Appl. No.: 19/451,614

(22) Filed: Jan. 16, 2026

Related U.S. Application Data
(63) Continuation of application No. 17/653,314, filed on Mar. 3, 2022, now Pat. No. 12,558,472.
(60) Provisional application No. 63/157,014, filed on Mar. 5, 2021.

(51) Int. Cl. A61M 1/00

(52) U.S. Cl. CPC A61M 1/62; A61M 1/63; A61M 1/73; A61M 2205/35; A61M 2205/8206; A61M 2210/1089

(57) ABSTRACT

Examples relate to devices, systems, and methods for fluid collection, such as fluid. A portable fluid collection system may include a fluid collection container, a pump in fluid communication with the fluid collection container, a battery operatively coupled to the pump, and a pack. The pack may be sized and dimensioned to include at least the pump, the battery, and the fluid collection container therein. The pump may be configured to pull a vacuum on an interior region of the fluid collection container effective to draw fluid from a fluid collection device into the fluid collection container.

### CROSS-REFERENCE TO RELATED APPLICATIONS

This application a continuation of U.S. application Ser. No. 17/653,314 filed on Mar. 3, 2022, which claims priority to U.S. Provisional Patent Application No. 63/157,014 filed on Mar. 5, 2021, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections. Conventional fluid collection devices also may be limited to use when a patient is confined to a bed in a supine position.

Thus, users and manufacturers of fluid collection devices continue to seek new and improved devices, systems, and methods to collect fluid.

### SUMMARY

Embodiments disclosed herein are related to fluid collection devices and methods of using fluid collection devices. In an embodiment, a portable fluid collection system is disclosed. The fluid collection system may include a fluid collection container, a pump in fluid communication with the fluid collection container, a battery operatively coupled to the pump, and a pack sized and dimensioned to include at least the pump, the battery, and the fluid collection container therein. The pump may be configured to pull an at least partial vacuum on an interior region of the fluid collection container effective to draw fluid from a fluid collection device into the fluid collection container.

In an embodiment, a portable fluid collection system may include a fluid collection device, a first tube in fluid communication with the fluid collection device, a pump in fluid communication with the fluid collection container, a battery operatively coupled to the pump, and a pack configured to hold at least the pump, the battery, and the fluid collection container therein. The fluid collection device may be configured to be positioned at least proximate to a urethra of a user. The pump may be configured to pull an at least partial vacuum on an interior region of the fluid collection container effective to draw fluid from the fluid collection device through the first tube into the fluid collection container.

In an embodiment, a method of assembly a portable fluid collection system is disclosed. The method may include positioning a fluid collection device proximate to a urethra of a user. The method also may include fluidly coupling the fluid collection device to the fluid collection container with a first tube. The method may further include placing a pump in fluid communication with the fluid collection container and detachably coupling a pack to a wheelchair. The pump may be configured to pull an at least partial vacuum on the interior region of the fluid collection container effective to draw fluid from the fluid collection device through the first tube into the fluid collection container. In some embodiments, the pack includes at least the pump, a battery, and the fluid collection container therein.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a portable fluid collection system, according to an embodiment.

FIG. 2A is a rear isometric view of a portable fluid collection system, according to an embodiment.

FIG. 2B is a schematic view of a portable fluid collection system, according to an embodiment.

FIG. 2C is a front isometric view of the portable fluid collection system of FIG. 2A and includes a fluid collection device, according to an embodiment.

FIG. 2D is a rear schematic view of a fluid collection system coupled to a wheelchair, according to an embodiment.

FIG. 2E is a rear isometric view of a portable fluid collection system, according to an embodiment.

FIG. 3A is an isometric view of a lower compartment of a portable fluid collection system, according to an embodiment.

FIG. 3B is a top view of the portable fluid collection system of FIG. 3A.

FIG. 4A is an isometric view of a fluid collection container, according to an embodiment.

FIG. 4B is a cross-sectional view of the fluid collection container of FIG. 4A.

FIG. 5 is a schematic view of a sensor, according to an embodiment.

FIG. 6 is a block diagram of a controller of a fluid collection system, according to an embodiment.

FIG. 7A is a schematic view of an indicator, according to an embodiment.

FIG. 7B is a schematic view of an indicator display on a smartphone, according to an embodiment.

FIG. 8A is a block diagram of a filter system in a fluid collection system, according to an embodiment.

FIG. 8B is an isometric view of a portable fluid collection system, according to an embodiment.

FIG. 9 is a rear isometric view of a fluid collection system including an alternator coupled to a wheelchair, according to an embodiment.

FIG. 10 is a flow diagram of a method for assembling a portable fluid collection system, according to an embodiment.

### DETAILED DESCRIPTION

Embodiments disclosed herein are related to portable fluid collection systems and related methods. Many users of fluid collection devices are over 65 years old with limited mobility, often relying on wheelchairs as a primary mode of transportation. Many users also spend a significant amount of their day in a seated or supine position. Users and caregivers, then, are benefited from a fluid collection system that may be both discrete and mobile, allowing users to use the fluid collection system to collect fluid both at home and on the go.

In many embodiments described herein, a fluid collection system may be relatively compact and configured to be portable. Embodiments of the fluid collection systems described herein are mobile and discreet, allowing a user to participate in social activities without alerting others to the incontinence of the users. For example, the fluid collection systems may include a pack configured to hold at least a pump of a fluid collection system therein to obscure the pump from view and/or dampen noise outside the fluid collection system. The fluid collected in the fluid collection systems described herein also may be stored in a fluid collection container that is obscured by the pack from view outside the fluid collection system and/or obscures the fluid held in the fluid collection container.

In some embodiments, the fluid collection system also includes a fluid collection device configured to be positioned at least proximate to a urethra of a user and a first tube in fluid communication with the fluid collection device where the pump may be configured to pull an at least partial vacuum on an interior region of the fluid collection container effective to draw fluid from the fluid collection device through the first tube into the fluid collection container.

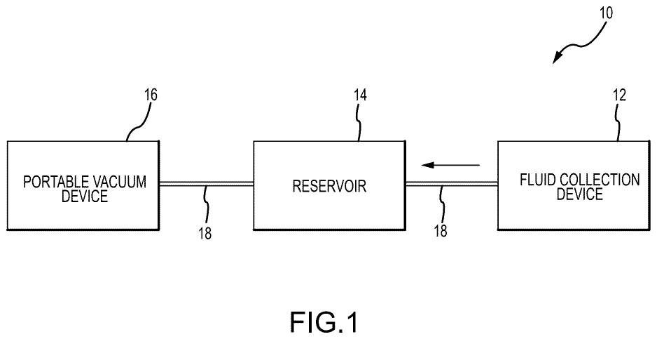

FIG. 1 is a block diagram of a fluid collection system 10, according to an embodiment. The fluid collection system 10 may be included in embodiments of fluid collection systems described herein. The system 10 includes a fluid (e.g., urine) collection device 12 (e.g., any of the fluid collection assemblies disclosed herein), a fluid collection container 14 (or reservoir), and a pump 16 (or portable vacuum device). The fluid collection device 10, the fluid collection container 14, and the pump 16 may be fluidly coupled to each other via one or more tubes 18. For example, fluid collection device 10 may be operably coupled to one or more of the fluid collection container 14 or the pump 16 via the tube 18. In some embodiments, the pump 16 may be coupled directly to the fluid collection container 14. Fluid (e.g., urine or other bodily fluids) collected in the fluid collection device 10 may be removed from the fluid collection device 10 via the tube 18 coupled to the fluid collection device 12. Suction force may be introduced into the chamber of the fluid collection device 12 via the inlet of the tube 18 responsive to suction (e.g., vacuum) force applied at the outlet of the tube 18.

The suction force may be applied to the outlet of the tube 18 by the pump 16 either directly or indirectly. The suction force may be applied indirectly via the fluid collection container 14. For example, the outlet of the tube 18 may be disposed within or fluidly coupled to an interior region of the fluid collection container 14 and an additional tube 18 may extend from the fluid collection container 14 to the pump 16. Accordingly, the pump 16 may apply suction to the fluid collection device 12 via the fluid collection container 14. The suction force may be applied directly via the pump 16. For example, the outlet of the tube 18 may be disposed within the pump 16. An additional tube 18 may extend from the pump 16 to a point outside of the fluid collection device 12, such as to the fluid collection container 14. In such examples, the pump 16 may be disposed between the fluid collection device 12 and the fluid collection container 14.

The fluid collection container 14 may be sized and shaped to retain a fluid therein. The fluid collection container 14 may include a bag (e.g., drainage bag), a bottle or cup (e.g., collection jar), or any other enclosed container for storing bodily fluid(s) such as fluid. In some examples, the tube 18 may extend from the fluid collection device 12 and attach to the fluid collection container 14 at a first point therein. An additional tube 17 may attach to the fluid collection container 14 at a second point thereon and may extend and attach to the pump 16. Accordingly, a vacuum (e.g., suction) may be drawn through fluid collection device 12 via the fluid collection container 14. Fluid, such as fluid, may be drained from the fluid collection device 12 using the pump 16.

The pump 16 or vacuum source may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

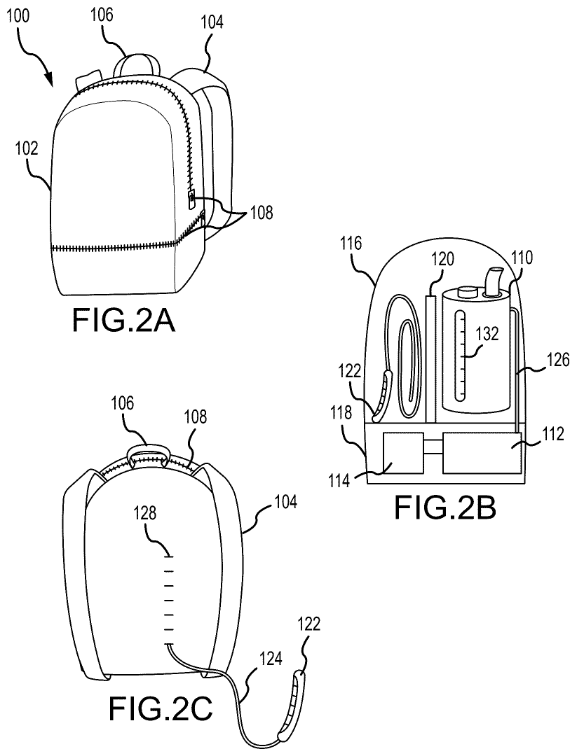

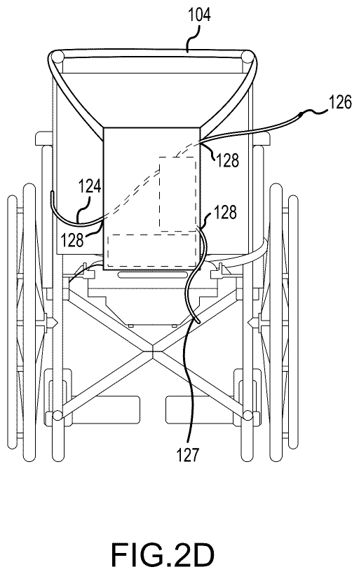

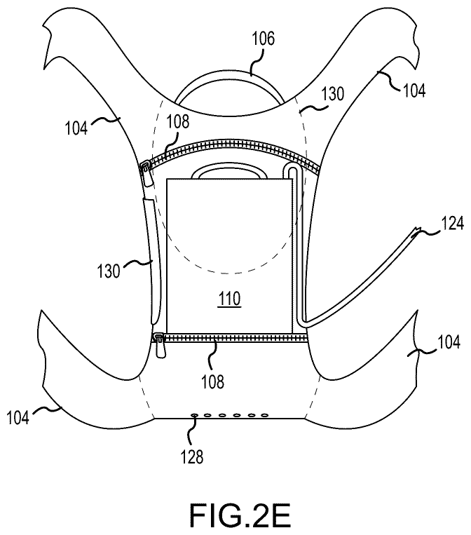

Many embodiments of fluid collection systems described herein are configured to be worn by a user, positioned on a surface such as a table, and/or securable or mountable to a wheelchair. Turning to FIG. 2A, a fluid collection system 100 is shown. The fluid collection system 100 may include a pack 102 that may be worn by a user and/or a caregiver. In some embodiments, the pack 102 may include one or more straps 104 to couple the pack 102 to at least one of the user, one or more wheelchair handles, a bed, or a table. The pack 102 may also be set on a shelf or table or mounted to a wheelchair. In some embodiments, an outer surface of the pack 102 may include a hook and loop fastener that may couple directly to the fabric of a wheelchair, bedding, or clothing. The one or more straps 104 may be configured to be worn on a shoulder or to detachably secure, mount, or hang from a stationary or mobile structure (e.g., a wheelchair) and support the fluid collection system 100. In an embodiment, the one or more straps 104 may include one or more fasteners configured to adjustably secure the strap 104, such as at least one of buckles, clips, and/or hook and loop fastener materials. The strap 104 may be constructed of an elastic material that may stretch to fit over the handles of a wheelchair. The pack 102 may include a handle 106 for carrying the pack 102. In some embodiments, the handle 106 may be elastic to span the handles or headrest of a wheelchair. In some embodiments, the pack 102 may include at least one of a backpack, handbag, purse, or shoulder bag.

In some embodiments, the pack 102 may include a fastening seam 108, such as a hook and loop, snap, or zipper seam. In some embodiments, the pack 102 may include more than one compartments, each compartment being able to be accessed by a fastening seam. Thus, the pack 102 may be closeable for discretion of the fluid collection system 100 and may be used to hold other items securely. Whether mounted to a wheelchair or worn by a user, the fluid collection system 100 may allow a user to discretely use and/or transport the fluid collection system 100 with the pack 102 that holds at least one component of a fluid collection system 100 therein. For example, the pack 102 may be sized and dimensioned to hold at least a fluid collection container 110, a pump 112, and a battery 114 (shown in FIG. 2B) therein.

Referring now to FIG. 2B, in some embodiments, the pack 102 may include an upper compartment 116 and a lower compartment 118. The upper compartment 116 may be configured to include at least the fluid collection container 110 and the lower compartment 118 may include at least the pump 112 and the battery 114. The battery 114 may be operably coupled to the pump 112 and described in more detail below. In some embodiments, the upper compartment 116 and lower compartment 118 may be separated by a moisture resistant material. In some embodiments, a fastening seam 108 may be able to completely separate the upper compartment 116 and the lower compartment 118. The upper compartment 116 may include pockets, sub-compartments, and/or a divider 120 configured to separate the upper compartment 116. In some embodiments, the divider 120 may be constructed of a rigid material such as a plastic or metal and may include a foam coating. In other embodiments, the divider 120 may be a foam or other suitable soft material. In some embodiments, the upper compartment 116 may be configured to hold at least a fluid collection device 122 and/or other items for storage within the pack 102. In other embodiments, the fluid collection device 122 may be stored in an external pocket for more convenient access.

In some embodiments, the divider 120 may be constructed of a moisture resistant material. The moisture resistant material may include a vinyl, woven fabrics, polytetrafluoroethylene (Teflon®), latex, a coated fabric, a hybrid synthetic material, or other suitable natural or synthetic material. In some embodiments, an upper portion of the pack 102 may be constructed of a first material and a lower portion of the pack 102 may be constructed of a second material. The second material may be waterproof or include a rubber or latex material or composite.

Referring now to FIG. 2C, in some embodiments, the fluid collection device 122 may be configured to be positioned at least proximate to a urethra of a user. While the fluid collection device 122 shown in FIG. 2C includes a female urine collection device, the fluid collection device 122 may instead include a male urine collection device. PCT International Application No. PCT/US2019/029616, for example, describes various embodiments of both male and female fluid collection devices that may be used in any of the embodiments disclosed herein, the disclosure of which is incorporated in its entirety by this reference. Moreover, the fluid collection device 122 may be interchangeable in the fluid collection system 100 between different types, varieties, and sizes of male or female fluid collection devices. Generally, the fluid collection device 122 may include a surface sized to be positioned proximate or adjacent to the urethra and configured to wick urine or other fluids away from the user. Urine or other fluids may be wicked from the surface to a reservoir in the urine collection device 122.

The fluid collection system 100 may also include a first tube 124 in fluid communication with an interior region (e.g. reservoir) of the fluid collection device 122 and an interior region of the fluid collection container 110. The first tube 124 may be positioned between the fluid collection device 122 and the fluid collection container 110. The fluid collection system 100 also may include a second tube 126 (shown in FIG. 2B) that may provide fluid communication between the pump 112 and the interior region of the fluid collection container 110. In some embodiments, the pump 112 may be coupled directly to the fluid collection container 110, and the second tube 126 may be absent from the fluid collection system 100. The tubes 124, 126 may include a flexible tube. In some embodiments, at least a portion of the first tube 124 may be substantially opaque, thereby inhibiting viewing of the fluid within the first tube 124. In some embodiments, the pack 102 may include at least one aperture 128 where the tubes 124, 126 may traverse from an exterior to an interior of the pack 102. The aperture 128 may be placed in any suitable location on the pack 102.

Referring to FIG. 2D, in some embodiments, a series of apertures 128 may be included such that tubes, cords, or components may access the interior of the pack 128 as required or desired to couple to the components of the fluid collection system 100. In some embodiments, the fluid collection container 110 may include a drain tube 127 to remove fluid from the fluid collection container 110. Fluid collected in the fluid collection container 110 may be emptied through the drain tube 127. In some embodiments, the drain tube 127 may include a valve (not shown) that may be opened to empty the fluid collection container 110 directly or a hand pump may be coupled to the first tube 124 and/or the second tube 126 to apply a pressure to the fluid for more rapid disposal. In some embodiments, the pump 112 may include a selector. The selector may configure the pump such that it is selectably configured to couple to the second tube 126 to pull a vacuum on the interior region of the fluid collection container 110 effective to draw fluid from the fluid collection device 122 or couple to the drain tube 127 to draw fluid from the fluid collection container 110. Thus, in other words, the first tube 124, the second tube 126, and the drain tube 127 may be selectively coupled to the pump 112 to either fill or drain the fluid collection container 110.

Referring to FIG. 2E, in some embodiments, the pack 102 may include a rigid reinforcement structure 130. The rigid reinforcement structure 130 may be a single component that includes a rigid material such as a plastic or metal. In some embodiments, the structure 130 may include more than one rigid components that may strengthen the pack 102 and/or protect the components of the fluid collection system 100 such as the battery 114 or the pump 112. In some embodiments, the structure 130 may include an exoskeleton or external frame having a rigid and/or durable structure. In other embodiments, the structure 130 may be included within the material of the pack 102 or be included as the divider 120.

Referring back to FIG. 2B, the fluid collection system 100 includes the fluid collection container 110. The fluid collection container 110 may include an interior region that stores fluid received from the fluid collection device 122 via the first tube 124. The fluid collection container 110 may be opaque or clear according to different embodiments and may include a generally rectangular front or rear profile or be cylindrical as shown in FIG. 2B. The fluid collection container 110 may be reusable and dishwasher safe, and may include a generally rigid material such as polycarbonate, plastic, rubber, metal, glass, combinations thereof, or any other suitable materials. The fluid collection container 110 may be sized and dimensioned to fit within the upper compartment 116 of the pack 102. In some embodiments, the fluid collection container 110 may include a transparent viewing window 132. The viewing window 132 may include markings to indicate a fluid level within the fluid collection container 110.

The pump 112 may be in fluid communication with the interior region of the fluid collection container 110 and may be configured to pull at least a partial vacuum on the interior region of the fluid collection container 110 effective to draw the fluid from the fluid collection device 122 through the first tube 124 into the fluid collection container 110. In some embodiments, the pump 112 may be coupled directly to the fluid collection container 110, or the tube 124 may fluidly couple the pump 112 with the interior region of the fluid collection container.

The pump 112 may include one or more of a manual vacuum pump, and electric vacuum pump, a diaphragm pump, a centrifugal pump, a displacement pump, a magnetically driven pump, a peristaltic pump, or any pump configured to produce a vacuum. For example, the pump may include an air media diaphragm pump having a minimum pumping speed of 25 ml/second. In some embodiments, the pump 112 includes a variable speed pump and/or a continuous pump. For example, the pump 112 may include a variable speed pump. The pump 112 may provide a vacuum or suction to remove fluid from the fluid collection device 122. In some examples, the pump 112 may be powered by one or more batteries 114 operatively coupled to the pump. In some embodiments, the battery 114 may include a lithium ion battery. In some embodiments, the battery 114 may be alkaline or rechargeable. In some examples, the pump 112 and/or the battery 114 may be sized and shaped to fit within the lower compartment 118 of the pack 102. For example, the pump 112 may include one or more miniaturized pumps or one or more micro pumps. The pump 112 may include one or more of a switch, a button, a plug, a remote, or any other device suitable to activate the pump 216.

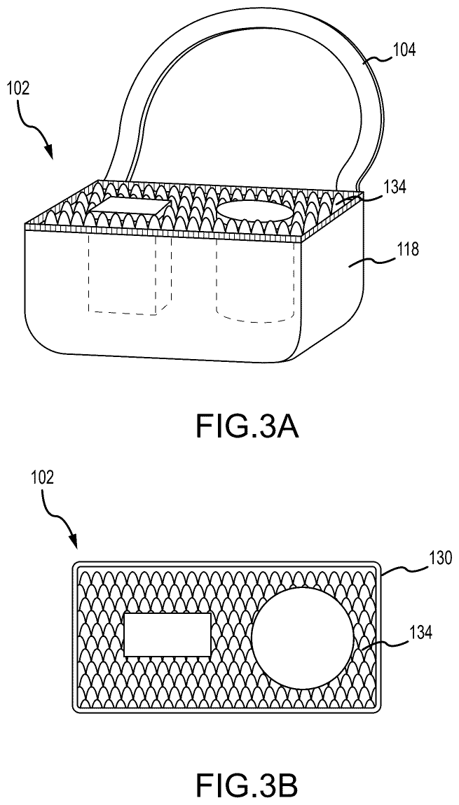

Referring now to FIGS. 3A-3B, the pack 102 may include a foam material 134 including at least one slot 135 where the pump 112 and/or the battery 114 may be located. FIGS. 3A-3B show an embodiment of the lower compartment 118 of the pack 102 separate from the upper compartment 116. In some embodiments, the foam material 134 may be configured to dampen the operational sound of the pump 112 and secure the pump 112 and/or the battery 114, and/or other components within the pack 102. The foam material 134 may include sound-deadening foam. Sound-deadening foam may include egg crate style foam constructed of open cell melamine, polyurethane, or other suitable foam material. The foam material 134 may include a composite of synthetic or natural materials. In addition to abating sound, the foam materials 134 may minimize operational vibrations of the fluid collection system 100. The one or more dividers 120 or foam material 134 may be positioned underneath and/or around at least a portion of the pump 112 to minimize vibrations and sounds emanating from the pump 112. In some embodiments, the foam material 134 may be positioned in the upper compartment 116 and/or the lower compartment 118 of the pack 102.

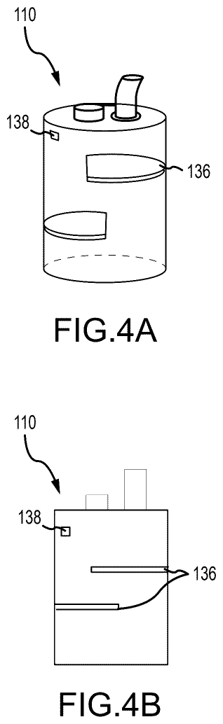

Referring now to FIGS. 4A-4B, the fluid collection container 110 may include at least one baffle 136 within an interior thereof. The at least one baffle 136 may be configured to mitigate fluid movement within the fluid collection container 110. Sound dampening of the fluid collection system 100 may be improved by the at least one baffle 136 within the interior region of the fluid collection container 110. The at least one baffle 136 may reduce the sloshing effect of fluid within the fluid collection container 110. The at least one baffle 136 may include a semicircular shape coupled to an outer wall of the interior region of the fluid collection container 110. Other suitable shapes of the baffle 136 may be included. In some embodiments, the baffle 136 may include a series of ridges applied to an interior surface of the fluid collection container 110. The at least one baffle 136 may be configured about halfway from the bottom of the fluid collection container 110 to mitigate movement of fluids. In some embodiments, several baffles 136 may be distributed throughout the interior of the fluid collection container 110. The fluid collection container may include 2-4 baffles in some embodiments.

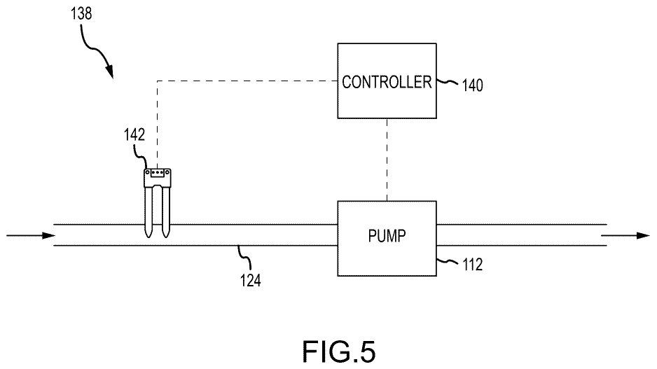

The fluid collection system 100 may also include a sensor 138 and a controller 140 (shown in FIG. 5). The sensor 138 may be coupled to the fluid collection container 110 and configured to detect a property relating at least to a volume of the fluid in the fluid collection container 110. In some embodiments, the sensor 138 may be located or positioned at an inlet (either interior or exterior) of the fluid collection container 110 for the first tube 124 or the second tube 126. In some embodiments, the sensor 138 may include an ultrasonic sensor, a laser sensor, or an ultraviolet (UV) sensor configured to provide a continuous or periodical feedback of the property relating at least to a volume of the fluid. In some embodiments, the sensor 138 may include a level transmitter configured to detect a level of the fluid in the fluid collection container 110. The sensor 138 may include a non-contact fluid sensor such as a capacitive sensor, an inductive sensor, a gravimetric sensor, or a mechanical float. The sensor 138 may be coupled to the fluid collection container 110 with at least a portion of the sensor 138 inside the interior region of the fluid collection container 110. In some embodiments, the sensor 138 may be positioned at a top of the fluid collection container 110 and pointed downwards where the fluid collects in the interior region of the fluid collection container 110.

Referring now to FIG. 5, in some embodiments, the sensor 138 may be positioned along and/or within the first tube 124, according to an embodiment. The sensor 138 may include a flow sensor coupled to the first tube 124 or the fluid collection device 122. In some embodiments, the pump 112 may operate based on a property related to fluid flow in the first tube 124. In some embodiments, the sensor 138 may be a moisture sensor 142. In some embodiments, the moisture sensor 142 may be a wet/dry sensor. The moisture sensor 142 may be operatively coupled to the pump 112 via the controller 140, where the pump 112 may be configured to operate only when required to pump the fluid from the fluid collection device 122 to the fluid collection container 110. The moisture sensor 142 may extend the battery life and or operating life of the pump 112 due to the lower duty cycle of the pump running and also adds a level of noise reduction from the pump 112 not constantly running. In some embodiments, the pump 112 may operate at a low speed until the sensor 138 or other sensor detects fluid passing through the first tube 124 into the fluid collection container 110, when the pump 112 then adjusts to a higher speed to prevent wetting or pooling of fluid at the fluid collection device 212. In some embodiments, the sensor 138 may be located within the first tube 124 or a pump housing. In some embodiments, the moisture sensor 142 may be included in the fluid collection device 122. The moisture sensor 142 may be coupled to the controller 140.

In some embodiments, the controller 140 may be configured to communicate with the sensor 138 through a wired or wireless connection. In some embodiments, the sensor 138 may include the controller 140. In some embodiments, when the moisture sensor 142 is wetted, the controller 140 initiates the pump 112 and the pump may run for a predetermined amount or time or until the moisture sensor 142 is no longer wetted.

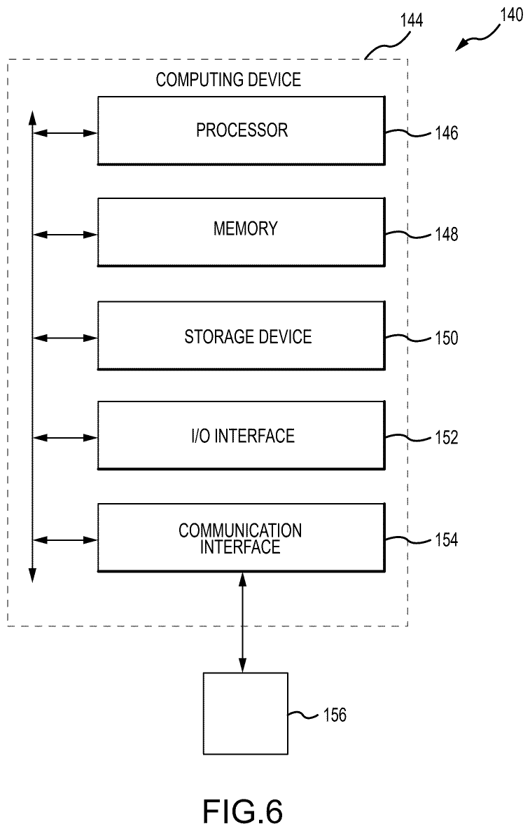

Referring now to FIG. 6, in an embodiment, the controller 140 may include a printed circuit board (PCB) equipped with erasable programmable read-only memory (EPROM) for memory of at least data collected by the sensor 138. The controller 140 may be configured to calculate a level or volume of fluid in the fluid collection container 110 or a volumetric flow rate or presence of fluid within the first tube 124. The controller 140 may be configured to send notifications or alerts to other electronic devices. For example, the controller 140 may be configured to send notifications or alerts to an indicator at a selected radio frequency, via BLUETOOTH, or via WI-FI to another electronic device, such as a mobile phone of the user or caregiver. The controller 140 may be powered by an external or internal battery, such as a rechargeable battery or the battery 114.

The controller 140 may include at least one computing device 144, according to an embodiment. The at least one computing device 144 may be an exemplary computing device that may be configured to perform one or more of the acts described above. The computing device 144 may include at least one processor 146, memory 148, a storage device 150, an input/output (“I/O”) device/interface 152, and a communication interface 154. While an example computing device 144 is shown in FIG. 6, the components illustrated in FIG. 6 are not intended to be limiting of the controller 140 or computing device 144. Additional or alternative components may be used in some examples. Further, in some examples, the controller 140 or the computing device 144 may include fewer components than those shown in FIG. 6.

In some examples, the processor 146 may include hardware for executing instructions (e.g., instructions for carrying out one or more portions of any of the methods disclosed herein), such as those making up a computer program. For example, to execute instructions, the processor 146 may retrieve the instructions from an internal register, an internal cache, the memory 148, or a storage device 150 and decode and execute them. In some examples, the processor 146 may be configured (e.g., include programming stored thereon or executed thereby) to carry out one or more portions of any of the example methods disclosed herein.

In some examples, the processor 146 may be configured to perform any of the acts disclosed herein or cause one or more portions of the computing device 144 or controller 140 to perform at least one of the acts disclosed herein. Such configuration can include one or more operational programs (e.g., computer program products) that are executable by the at least one processor 146. For example, the processor 146 may be configured to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a fluid is sensed in the fluid collection device 122, and/or automatically transmit an alert when a change or recharge of battery is suggested.

The at least one computing device 144 may include at least one memory storage medium (e.g., memory 148 and/or storage device 150). The computing device 144 may include memory 148, which is operably coupled to the processor 146. The memory 148 may be used for storing data, metadata, and programs for execution by the processor 146. The memory 148 may include one or more of volatile and non-volatile memories, such as Random Access Memory (RAM), Read Only Memory (ROM), a solid state disk (SSD), Flash, Phase Change Memory (PCM), or other types of data storage. The memory 148 may be internal or distributed memory.

The computing device 144 may include the storage device 150 having storage for storing data or instructions. The storage device 150 may be operably coupled to the at least one processor 146. In some examples, the storage device 150 may comprise a non-transitory memory storage medium, such as any of those described above. The storage device 150 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 150 may include removable or non-removable (or fixed) media. Storage device 150 may be internal or external to the computing device 144. In some examples, storage device 150 may include non-volatile, solid-state memory. In some examples, storage device 150 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 148 and/or storage device 150 (e.g., memory storage medium(s)) may store one or more databases thereon.

In some examples, one or more of a history of the volume of the fluid in the fluid collection container 110, a trend of the volume of the fluid in the fluid collection container 100, a history of a fluid collection device 122 replacement, and/or a history of battery 114 replacement or recharging may be stored in a memory storage medium such as one or more of the processor 146 (e.g., internal cache of the processor), memory 148, or the storage device 150.

The computing device 144 also includes one or more I/O devices/interfaces 152, which are provided to allow a user to provide input to, receive output from, and otherwise transfer data to and from the computing device 144. These I/O devices/interfaces 152 may include a mouse, keypad or a keyboard, a touch screen, camera, optical scanner, network interface, web-based access, modem, a port, other known I/O devices or a combination of such I/O devices/interfaces 152. The touch screen may be activated with a stylus or a finger.

The I/O devices/interfaces 152 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 152 are configured to provide graphical data to a display for presentation to a user. The graphical data may be representative of one or more graphical user interfaces and/or any other graphical content as may serve a particular implementation.

The computing device 144 may further include a communication interface 154. The communication interface 154 may include hardware, software, or both. The communication interface 154 may provide one or more interfaces for communication (such as, for example, packet-based communication) between the computing device 144 and one or more networks. For example, communication interface 154 may include a network interface controller (NIC) or network adapter for communicating with an Ethernet or other wire-based network or a wireless NIC (WNIC) or wireless adapter for communicating with a wireless network, such as a WI-FI.

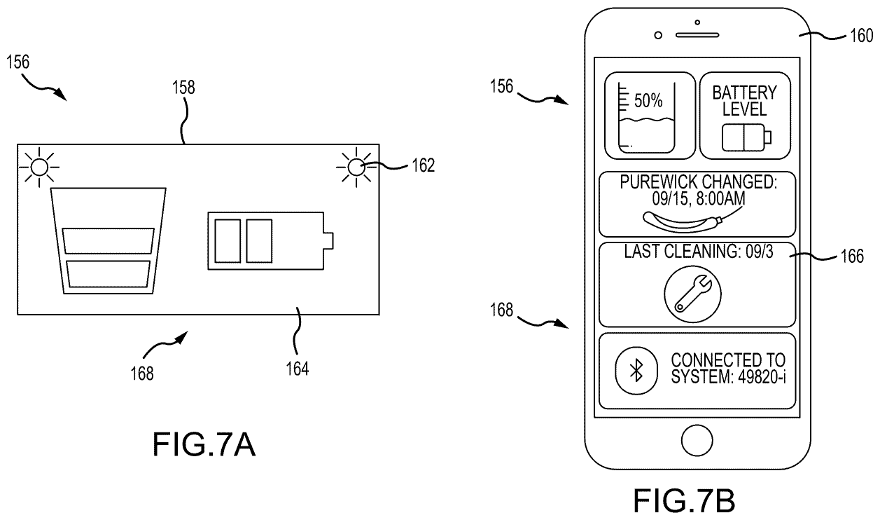

Referring now to FIGS. 7A-7B, in some embodiments, the computing device 144 may communicate with an indicator 156 that may include an indicator panel 168, a smart phone 160 of the user and/or the caregiver, and/or a computer device of a healthcare system. In an embodiments, the controller 140 may be configured to transmit a signal including or encoding a property related to the fluid collection system 100 or a status of the fluid collection system 100 to the indicator 156. In some embodiments, the indicator 156 may include at least one of an alarm or alert 162, a digital display 164, a smartphone interface 166, or any other suitable indicator panel 168, which may be coupled to the wheelchair of a user, or the pack 102.

In some embodiments, the indicator 156 may include an indication of at least one of a battery 114 status, an operating status of the pump 112, when the property relating to at least to the volume of the fluid in the fluid collection container 110 detected by the sensor 138 indicates the volume of the fluid in the fluid collection container 110 has reached or exceeded a predetermined volume. In an embodiment, the indication may include a volumetric or mass flow rate of the fluid in the first tube 124 or the presence of a fluid in the first tube 124, the fluid collection device 122, or the fluid collection container 110. For example, based on data from the sensor 138, the controller 140 may wirelessly transmit an alert to a smartphone 160 of a caregiver that the fluid in the fluid collection device 110 is at a predetermined distance (such as about 1 inch) from the sensor 138 and emptying of the fluid collection container 110 is recommended. In some embodiments, the controller 140 may wirelessly transmit alerts and selected frequencies, such as selected time and/or volume intervals. The controller 140 may wirelessly transmit an alert to the electronic device of the user or the caregiver when a battery powering at least one of the controller 140, the indicator 156, or the pump 112 is low. The controller 140 may wirelessly transmit an alert to the electronic device of the user or the caregiver when cleaning or replacement of the fluid collection device 122 or other component may be due. In some embodiments, a system parameter may be indicated. The parameter may include at least one of a property relating at least to a volume of the fluid in the fluid collection container, a battery status, a fluid collection device or fluid collection system cleaning schedule, and a connectivity status with an electronic device or smartphone 160.

In some embodiments, the indicator 156 may issue an alarm and/or alert 162 to the user or caregiver. The alert may include a light, a sound, or a message on the digital display 164 or smartphone interface 166. In an embodiment, the indicator may transmit a kinesthetic communication through a seating pad or smartphone 160 to indicate a system parameter has reached or exceeded a predetermined condition. The kinesthetic communication may continue until acknowledged or may be transmitted intermittently.

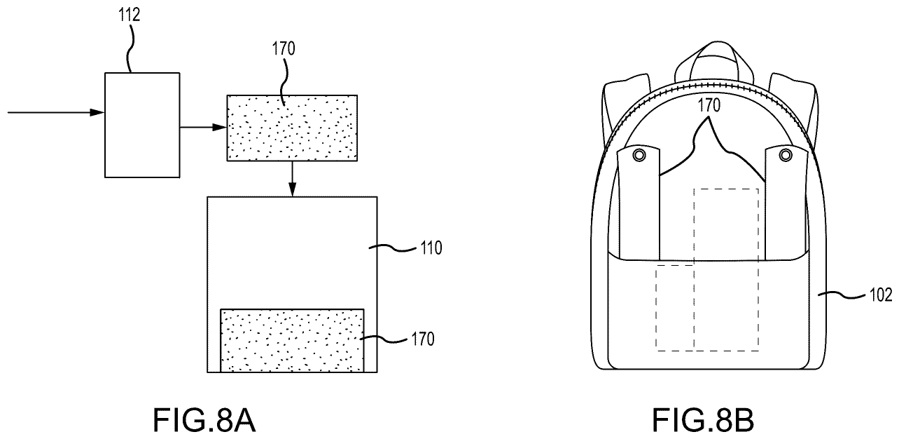

Referring now to FIGS. 8A-8B, in some embodiments, the fluid collection system 100 may include one or more odor filters 170 configured to at least partially filter or neutralize an odor of the fluid. The filter 170 may be configured to neutralize odor of the air being pulled from the interior region of the fluid collection container 110 by the pump 112. In some embodiments, the filter 170 may be positioned between at least a portion of the second tube 126 and a portion of the interior region of the fluid collection container 110 such that air being pulled from the interior region of the fluid collection container 110 may be filtered before or as the air enters the second tube 126. In some embodiments, the filter 170 may be positioned on an exhaust vent on the pump 112. In some embodiments, a filter 170 may be positioned both at the exhaust vent on the pump 170 and before air enters the second tube 126. The filter 170 may include an odor absorbing filter and/or a hydrophobic filter configured to prevent or minimize fluid from the fluid collection container 110 being pulled into the pump 112.

In some embodiments, the filter 218 may include an aromatherapy pack or an aromatherapy pack may be coupled proximate to the exhaust vent of the pump to produce a more pleasant smell. The filter may include baking soda or other composition that removes odor from the air and/or adds pleasant aroma to the air. In some embodiments, the filter 218 is absent and the pump 216 includes the aromatherapy pack removably coupled to the pump 216 proximate to the exhaust vent.

In other embodiments, the one or more odor filter 170 may be included on or within at least one of the fluid collection device 122, the pack 102, or the fluid collection container 110. The odor filter 170 may include activated charcoal filters placed or embedded within an interior of the pack 102 to mitigate odor from the fluid.

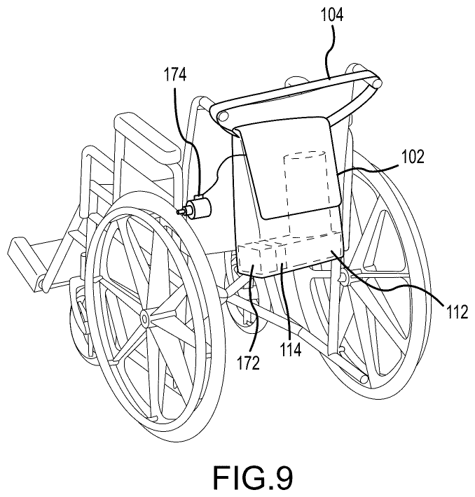

Referring now to FIG. 9, in some embodiments, the fluid collection system 100 may be powered by the battery 114 and may also include a second battery 172. The second battery may be the same type as the battery 114 and may be used when the battery 114 is at a low power status or charging. In some embodiments, the fluid collection system 100 may include an alternator 174 to charge the battery 114, the second battery 172, or power the pump 112. In some embodiments, the alternator 174 may be coupled to a wheel of the wheelchair. When the wheelchair is moved, the alternator may be coupled to the wheel to generate a current that may charge the battery 114 and/or power the pump 112. The alternator 174 may extend the life of the battery 114 in operation, charge the second battery 172, operate the pump 112 or other component of the fluid collection system 100, or charge both batteries 114, 172 simultaneously.

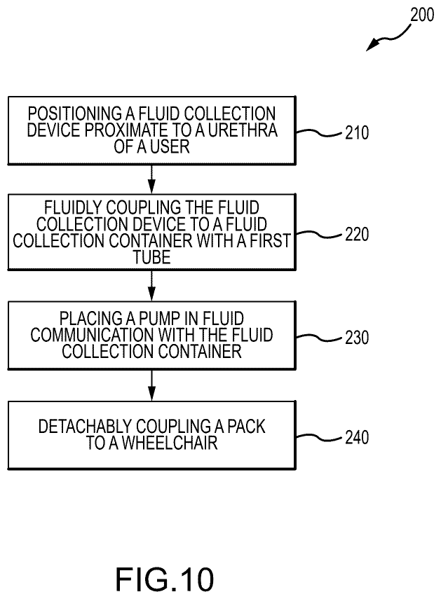

FIG. 10 is a flow diagram of a method 200 for assembling a portable fluid collection system, according to an embodiment. The method 200 includes an act 210 of positioning a fluid collection device proximate to a urethra of a user and an act 220 of fluidly coupling the fluid collection device to a fluid collection container with a first tube. The method 200 also includes an act 230 of placing a pump in fluid communication with the fluid collection container. The pump may be configured to pull a vacuum on the interior region of the fluid collection container effective to draw fluid from the fluid collection device through the first tube into the fluid collection container. The method 200 may also include an act 240 of detachably coupling a pack to a wheelchair. In some embodiments, the pack may include at least the pump, a battery, and the fluid collection container therein.

The method 200 may include assembling any of the fluid collection system embodiments described herein. For example, the act 240 of detachably coupling a pack to a wheelchair may include coupling one or more straps of the pack to one or more handles of the wheelchair. In some embodiments, the act 240 may further include coupling the fluid collection container within the pack. In some embodiments, coupling the fluid collection container within the pack includes positioning the fluid collection container inside a compartment within the pack. In some embodiments, the method 200 may further include an act of fluidly coupling the fluid collection container to the pump with a second tube.

The acts of the method of collecting fluids from a user described above are for illustrative purposes. For example, the acts of the method of collecting fluids from a user can be performed in different orders, split into multiple acts, modified, supplemented, or combined. In an embodiment, one or more of the acts of the method of collecting fluids from a user can be omitted from the method. Any of the acts of the method of collecting fluids from a user can include using any of the portable fluid collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A portable fluid collection system, comprising:
a fluid collection container;
a pump in fluid communication with the fluid collection container configured to pull an at least partial vacuum on an interior region of the fluid collection container effective to draw fluid from a fluid collection device through a tube and into the fluid collection container;
a battery operatively coupled to the pump;
a pack having an interior region sized and dimensioned to include at least the pump, the battery, and the fluid collection container therein, the pack including an aperture; and
one or more rigid members sized and dimensioned to fit within the interior region of the pack, the one or more rigid members configured and positionable within the pack to protect at least one of the pump or the battery,
wherein the tube traverses from an exterior to an interior of the pack through the aperture.

2. The portable fluid collection system of claim 1, wherein the one or more rigid members are positioned to at least partially encase at least one of the pump or battery when the fluid collection container, the pump, and the battery are positioned within the pack.

3. The portable fluid collection system of claim 1, further comprising a foam material at least partially encasing the pump and positioned between two opposing walls of the one or more rigid members.

4. The portable fluid collection system of claim 1, further comprising a foam material at least partially encasing the battery and positioned between two opposing walls of the one or more rigid members.

5. The portable fluid collection system of claim 1, wherein the fluid collection container is removably positioned between two opposing walls of the one or more rigid members.

6. The portable fluid collection system of claim 1, further comprising:
a foam material at least partially encasing the pump and positioned between two opposing walls of the one or more rigid members;
a foam material at least partially encasing the battery and positioned between two opposing walls of the one or more rigid members,
wherein the fluid collection container is removably positioned between two opposing walls of the one or more rigid members.

7. The portable fluid collection system of claim 1, wherein at least a portion of the fluid collection container is positioned over at least a portion of the battery.

8. The portable fluid collection system of claim 1, further comprising:
a foam material at least partially encasing the pump and positioned between two opposing walls of the one or more rigid members;
a foam material at least partially encasing the battery and positioned between two opposing walls of the one or more rigid members;
wherein the one or more rigid members are positioned to at least partially encase at least one of the pump or battery when the fluid collection container, the pump, and the battery are positioned within the pack;
wherein the fluid collection container is positioned between two opposing walls of the one or more rigid members; and
wherein at least a portion of the fluid collection container is positioned over at least a portion of the battery.

9. A portable fluid collection system, comprising:
a fluid collection container;
a pump in fluid communication with the fluid collection container configured to pull an at least partial vacuum on an interior region of the fluid collection container effective to draw fluid from a fluid collection device through a tube and into the fluid collection container;
a battery operatively coupled to the pump;
a pack having an interior region sized and dimensioned to include at least the pump, the battery, and the fluid collection container therein, the pack including an aperture, wherein the tube traverses from an exterior to an interior of the pack through the aperture; and
one or more rigid members sized and dimensioned to fit within the interior region of the pack, wherein the fluid collection container is removably positioned between two opposing walls of the one or more rigid members.

10. The portable fluid collection system of claim 8, wherein the one or more rigid members are positioned to at least partially encase at least one of the pump or battery when the fluid collection container, the pump, and the battery are positioned within the pack.

11. The portable fluid collection system of claim 10, further comprising a foam material at least partially encasing the pump and positioned between two opposing walls of the one or more rigid members.

12. The portable fluid collection system of claim 10, further comprising a foam material at least partially encasing the battery and positioned between two opposing walls of the one or more rigid members.

13. The portable fluid collection system of claim 8, wherein at least a portion of the fluid collection container is positioned over at least a portion of the battery.

14. The portable fluid collection system of claim 8, further comprising:
a foam material at least partially encasing the pump and positioned between two opposing walls of the one or more rigid members;
a foam material at least partially encasing the battery and positioned between two opposing walls of the one or more rigid members;
wherein the one or more rigid members are positioned to at least partially encase at least one of the pump or battery when the fluid collection container, the pump, and the battery are positioned within the pack; and
wherein at least a portion of the fluid collection container is positioned over at least a portion of the battery.

15. A portable fluid collection system, comprising:
a fluid collection container;
a pump in fluid communication with the fluid collection container configured to pull an at least partial vacuum on an interior region of the fluid collection container effective to draw fluid from a fluid collection device through a tube and into the fluid collection container;
a battery operatively coupled to the pump; and
a pack having an interior region sized and dimensioned to include at least the pump, the battery, and the fluid collection container therein, the pack including an aperture, wherein the tube traverses from an exterior to an interior of the pack through the aperture;
one or more rigid members sized and dimensioned to fit within the interior region of the pack; and
a foam material at least partially encasing the pump and positioned between two opposing walls of the one or more rigid members.

16. The portable fluid collection system of claim 15, wherein the fluid collection container is removably positioned between two opposing walls of the one or more rigid members.

17. The portable fluid collection system of claim 15, wherein the one or more rigid members are positioned to at least partially encase at least one of the pump or battery when the fluid collection container, the pump, and the battery are positioned within the pack.

18. The portable fluid collection system of claim 15, further comprising a foam material at least partially encasing the battery and positioned between two opposing walls of the one or more rigid members.

19. The portable fluid collection system of claim 15, wherein at least a portion of the fluid collection container is positioned over at least a portion of the battery.

20. The portable fluid collection system of claim 15, wherein:
the fluid collection container is removably positioned between two opposing walls of the one or more rigid members;
the one or more rigid members are positioned to at least partially encase at least one of the pump or battery when the fluid collection container, the pump, and the battery are positioned within the pack;
a foam material at least partially encases the battery and is positioned between two opposing walls of the one or more rigid members; and
at least a portion of the fluid collection container is positioned over at least a portion of the battery.
